(19) United States
(12) Patent Application Publication
(10) Pub. No.: US 2010/0317039 A1
(43) Pub. Date: Dec. 16, 2010

(54) MOLDING WITH EMBEDDED COUPLING PARTICLES FOR BIOMOLECULES

(76) Inventors: Natalie Salk, Hude (DE); Ingo Grunwald, Bremen (DE)

Correspondence Address: WEINGARTEN, SCHURGIN, GAGNEBIN & LEBOVICI LLP, TEN POST OFFICE SQUARE, BOSTON, MA, 02109, US

(21) Appl. No.: 12/791,200

(22) Filed: Jun. 1, 2010

(30) Foreign Application Priority Data
May 29, 2009 (DE) 10 2009 026 622.4

(51) Int. Cl. B32B 3/10; B29C 45/14; G01N 33/53; C12P 1/00; C12M 3/00; C11D 17/00

(52) U.S. Cl. 435/7.92; 428/147; 264/119; 436/525; 436/531; 435/41; 435/395; 435/7.1; 510/438

(57) ABSTRACT

The invention relates to a molding, comprising a matrix in a material, selected from the group consisting of metal, ceramic and polymer synthetic material, and coupling particles embedded in the matrix, wherein a proportion of the surface of the molding in a geometrical form or in a regular pattern and/or an area of the molding is completely or the entire surface of the molding is mechanically treated.

### CROSS REFERENCE TO RELATED APPLICATIONS

Not Applicable

### STATEMENT REGARDING FEDERALLY SPONSORED RESEARCH OR DEVELOPMENT

Not Applicable

### FIELD OF THE INVENTION

The invention relates to a molding, comprising a matrix in a material, selected from the group consisting of metal, ceramic and polymer synthetic material and coupling particles embedded in the matrix, wherein a proportion of the surface of the molding in geometrical form or in a regular pattern and/or an area of the molding is completely or the entire surface of the molding is mechanically treated.

In particular the invention relates to a molding, comprising a polymer synthetic material matrix and coupling particles embedded therein, wherein the molding is mechanically treated, so that a proportion of the particle is at least not covered by the synthetic material matrix or the surrounding metal or ceramic matrix, and wherein a proportion of the surface of the molding in a geometrical form or in a regular pattern and/or an area of the molding is completely or the entire surface of the molding is mechanically treated.

### BACKGROUND OF THE INVENTION

As an example of the introduction of active groups to surfaces, reference is made to WO 03030129, in which silanization takes place in order to obtain reactive amino groups.

For the coupling of complex biomolecules these usually have to be brought into a reaction with surface groups through cross-linkers (homo- or heterofunctional) (see e.g. Hermanson G., Bioconjugate Techniques, Academic Press, London, 1996).

If the biomolecules contain special groups (also referred to as tags), which because of their chemical structure are particularly suited to interaction with parts of a surface, then the point of binding of the biomolecule can be accurately controlled on the basis of the location of the tag on the molecule surface. An example of this is the so-called his-tags (sequence repetition of the amino acid histidine), which through complexing interactions react with nickel ions and bind the biomolecule to the surface. In the literature, however, reactive surface groups are also described, e.g. epoxy, hydrazine, isocyanate, catechol and azlactone groups, which can react directly for example with the hydroxyl, sulfhydryl amino or carboxyl groups of the biomolecules (see Hermannsson G., Bioconjugate Techniques, Academic Press, London, 1996).

The activation of the surfaces with these groups is often a chemically complicated process. Furthermore, for many applications base bodies have to be provided with a surface coating in a complicated manner or the surfaces have to be modified in another way, so that items which are set up for the coupling of biomolecules often have to be manufactured in multi-stage processes.

On top of this there is the fact that many activation methods from the state of the art—and this applies in particular to the relatively cheapest—are not very specific, so that often not only one kind of chemical or physical (active) binding group is made available. However, this leads to an often undesired non-specific binding behavior of the biomolecules to be attached, so that in turn a proportion of the biomolecules is not provided with the ideal alignment, the biomolecule is denatured due to the incorrect attachment or biomolecules other than the ones desired are attached.

It is also a problem that for various applications certain base materials are used by preference: these base materials provide the object with the surface to which the biomolecules are to be attached with e.g. certain mechanical basic properties. If it is desired to avoid a relatively complicated coating method, this means that for each base material its own binding chemistry for each (desired) biomolecule or at least each (desired) group of biomolecules must be developed.

This applies in particular also if changes are made to the composition of the base material, which could be as minor as a change to the coloring of the material.

On top of this, for a range of materials—and this applies also to a number of synthetic materials—a suitable binding chemistry to a large number of biomolecules has not yet been developed.

### BRIEF SUMMARY OF THE INVENTION

Against the background of the state of the art, the object of the invention was therefore to propose a system can be adapted which without great effort to desired basic requirements, such as for example mechanical stability or thermal conductivity, which at the same time but similarly without great effort is adapted to the binding requirements for the binding of desired biomolecules. The system should preferably allow corresponding objects to be produced cheaply and/or in the fewest possible work stages.

The invention also relates to a method for manufacturing such a molding and the use of such a molding as sensor, biochip, for diagnostic purposes for immunological and cell biology detection methods, as bioreactor or component of a bioreactor, for lab-on-a-chip applications, for cleaning mixtures containing biomolecules, for testing substances for impurities, for generation of or as a biocatalytic and/or bioactive surface, for cell culture purposes or for working with cultivated cells on surfaces, or for manufacturing an implant. Coupling of biomolecules to surfaces is currently performed by means of two main methods: in the first method the molecules are bound to the surface unaligned (physisorption). Here the molecules are adsorbed by means of electrostatic forces such as for example hydrogen bridge bonding, Van der Waals forces, dipole-dipole interactions or hydrophobic interaction forces. This form of binding is often of limited stability and binding energy. In addition the binding, because it is unaligned, often takes place in such a way that considerable proportions of the active groups are no longer available in the biomolecules for the intended reaction since, because of the binding position, they are sterically no longer accessible.

In the second method, the binding of the biomolecules takes place covalently via corresponding reactive groups on the surface (chemisorption). These reactive groups can, for example, be generated by the plasma activation methods, by active separation processes or by chemical reactions (reactive gases or liquids). Certain coatings can also be provided for, via which reactive groups are made available on the surface. In the literature a number of possible reactive groups are described. The most important of these are phosphate, primary amino, carboxyl, carbamide, thiole, or quaternary amino groups, as referred to for example in DE 3126551 A1, US 2004/0209269 A1 or WO 9852619.

### BRIEF DESCRIPTION OF THE DRAWINGS

FIG. 1 shows a schematic representation of a molding in accordance with the present invention;

FIG. 2 shows a photo-reactor mold part in accordance with the present invention;

FIG. 3 shows injection molded parts having 10% (by volume) glass balls in a polyethylene matrix;

FIG. 4 shows the surface of a molding having 10% (by volume) glass balls in a polyethylene matrix;

FIG. 5A shows a fluorescent microscope image of polyethylene moldings in accordance with Example 1 of the present invention in which the molding includes a silane functionalization;

FIG. 5B shows fluorescent microscope images of polyethylene moldings in accordance with Example 1 of the present invention in which the molding includes aminosilane-functionalized particles; and

FIG. 6 shows fluorescence microscopy image of the molding in accordance with Example 2 of the present invention.

### DETAILED DESCRIPTION OF THE INVENTION

This object is achieved by a molding, comprising a matrix in a material, selected from the group consisting of metal, ceramic and polymer synthetic material, and coupling particles embedded in the matrix, wherein a proportion of the surface of the molding in a geometrical form or in a regular pattern and/or an area of the molding is completely or the entire surface of the molding is mechanically formed.

In the sense of the present text, “matrix” means the material which essentially gives the molding its shape. Normally and preferably the molding predominantly consists of the matrix material, preferably ≧80%, more preferably ≧90%. The matrix material can basically also be a mixture of a number of different materials.

“Coupling particles” in the sense of the present text are particles that defer in the material composition from the matrix material. The person skilled in art will chose the form of the particles and the size of the particles dependent of the respective purpose. Preferably the size of the maximum diameter of the coupling particles is in the range on 10 nm to 50 μm, more preferred 50 nm to 10 μm and especially preferred 100 nm to 5 μm, each referred to the arithmetic average of the size contribution.

The particles may have anisotropic forms (like rods, needles, ovals etc.). The measurement of particle size is made by microscopic techniques. In this connection the person skilled in the art will chose the respective adequate methods for determining all particle sizes and their size contribution. It is preferred that the before mentioned arithmetic average size contribution is determined as follows: for the calculation are only those particles used that show in a light microscope a maximum diameter of ≧10 μm and those particle that show in a scanning electron microscope (SEM) a maximum diameter <10 μm and ≧100 nm and those particles that show in a transmission electron microscope a maximum diameter of <10 nm.

The person skilled knows that if non-spherical particles are examined under microscope the orientation of the single particle may play a role for the result of the respective maximum diameter (determined by microscope). However, when determining the arithmetic average it is preferred that only the maximum particle diameters are considered that have in fact been determined in the respective measurement. Certainly, this does not apply when non-spherical particles have been specifically brought in alignment. In this case the cut for the microscopic measurement has to be made so that the objective maximum diameter of the (aligned) particles can be measured by the utilized method of microscope.

It is further preferred that the particle material and the matrix material defer from each other clearly e.g. as particle material and matrix material are each chosen from a different of the following groups of material: metals/metal alloys, metal oxides, ceramics, polymers, especially polymer synthetic material, anorganic glasses.

A “molding” in the sense of the present text is a geometrical body, which is manufactured through a forming production process such as in particular injection molding, stamping, pinch-pointing, pressing and sintering, slip casting.

“Metal” in the context of this invention means pure metals and alloys.

The coupling particles must be formed according to the manufacturing method of the molding. Here it is preferable if the coupling particles have a higher melting point than the matrix material, in particular for methods in which the moldings are manufactured by melting or sintering of the matrix material (see also below).

Where the matrix material is essentially a ceramic material, a person skilled in the art must of course select the coupling particles in such a way that these do not become a component of the ceramic during the manufacturing process of the molding. This basically does not preclude ceramic coupling particles being embedded in a likewise ceramic matrix. It simply has to be ensured here that coupling particles and matrix can be spatially distinguished from one another.

Preferred manufacturing methods for moldings with a matrix in metal are powder metallurgy methods such as pressing or sintering, including but not restricted to metal powder injection molding, paste printing, embossing or printing.

Preferred manufacturing methods for moldings with a matrix in ceramic are pressing, sintering, ceramic injection molding, slip casting or printing, and freeze casting.

Under certain circumstances it may also be preferable for the inventive molding to be a multi-component body, and therefore consisting of different matrix material areas such as for example synthetic material, ceramic and/or metal. Particular preference in this connection is that only one of the matrix materials mentioned is mixed with coupling particles in an inventive multi-component molding.

The feature by which “the molding is mechanically treated”, in the sense of the invention, means that the molding, following its own manufacturing process, undergoes subsequent mechanical treatment in which material is removed from the molding. Preferred removal methods in the sense of this invention are grinding, milling and/or blasting, polishing, laser removal, (CO₂—) snow blasting. Here the treatment methods leave behind traces on the molding allowing the method used to be identified by a person skilled in the art.

The feature by which “a proportion of the surface of the molding in a geometrical form or in a regular pattern is mechanically treated”, means that a specific surface treatment is undertaken, which differs from random stress marks that may for example occur during usage. In this connection geometrical form preferably means classic geometrical forms such as circles, squares, rectangles, trapeziums, parallelograms, octahedrons, tetrahedrons, polyhedrons. Here these geometrical forms can also of course be fashioned in three dimensions, e.g. as wells. A regular pattern in the sense of the application means that the treated areas comprise repetitive shapes.

The feature by which the “entire surface or an area of the molding is completely mechanically treated” also distinguishes the inventive moldings from moldings which merely exhibit marks from usage.

The object outline above is preferably achieved by a molding, comprising a polymer synthetic material matrix and coupling particles embedded in it, wherein the molding is mechanically treated so that a proportion of the particles are at least partially not covered by the synthetic material matrix, and wherein a proportion of the surface of the molding in a geometrical form or in a regular pattern and/or an area of the molding is completely or the entire surface of the molding is mechanically treated.

A polymer synthetic material matrix in the sense of this text is a matrix of conventional polymers with repetitive identical or different subunits.

The feature by which the “entire surface or an area of the molding is completely mechanically treated” also distinguishes the inventive moldings from moldings which merely exhibit marks from usage.

The advantage of the inventive molding is in particular that materials with two basic functions can be combined with one another: firstly here it is a matter of the matrix (preferably the polymer synthetic material matrix), which provides the mechanical basic features and other features such as setting electrical and/or thermal conductivity and/or magnetic properties. Secondly it is a case of the coupling particles which, with regard to their surface, are adapted to the desired binding system. This makes it possible to always use the same kind of coupling particles for the binding of a particular target (bio)molecule, while the matrix in turn can be adapted to the other conditions of the planned use of the inventive molding. Here these components can be combined with each other as required over long periods.

In the state of the art, these various problems have often been solved by coating a base body, which—as already indicated above—calls for an additional work step. Finally through the inventive molding the coupling chemistry is to a large extent independent of the matrix and base material, although in actual fact only one material phase is needed, i.e. the molding can essentially be fashioned homogenously (with an even distribution of the coupling particles).

It is also advantageous that the preferred inventive moldings can be manufactured using common synthetic material manufacturing methods and in particular molding methods. One problem to be solved according to the invention was in this connection that, in normal manufacturing methods for moldings, the synthetic material matrix is liquefied. Since the in the context of the manufacturing process the coupling particles are dispersed in the liquid matrix, this normally leads to a complete inclusion of the particles in the synthetic material matrix, since the liquid synthetic material always fully encloses the particles because of compounding and surface effects. Metal and ceramic matrices often behave in a similar way, wherein after the forming of the molding a further sintering step follows metal powder or ceramic injection molding, in order to increase the cohesion of the molding. Embedded coupling particles such as metal particles in ceramic matrices or ceramic particles in metal matrices can likewise lead to improved material properties.

In particular, if it is a case of a homogenous distribution of the particles, the dispersion of the particles in the liquid synthetic material matrix usually lasts for a number of minutes. In the process a thin polymer skin forms on the particles, which essentially fully encloses the particles, which are ultimately positioned on the surface during the manufacturing process of the molding.

Through this inclusion the coupling particles would not be available for binding, since the surface of the molding is exclusively made up of matrix material. This problem is solved according to the invention through specific mechanical treatment of the molding. Here it may be advantageous to treat only certain areas of the molding, so that there are regions with a high concentration of exposed coupling particles, while in other areas, which have not been treated, practically no coupling particles are available on the surface. This allows optimal control of the location of the binding of the desired biomolecules to the molding. In this connection it is preferable that the matrix material in relation to the biomolecules to be bound is largely and more preferably completely inert.

The mechanical treatment offers further advantages, however, including in the event that the matrix material does not comprise any synthetic material: thus a suitably performed mechanical treatment leads to coupling particles being less strongly removed than the matrix material. This leads to an increase in the area available for coupling relative to the surface of the molding. Furthermore, the mechanical treatment allows the creation of areas of preferred binding on the surface of the molding. This takes place firstly in that in the area of the mechanical treatment a relatively increased binding area is made available, and secondly the mechanical treatment can be carried out in such a way that three-dimensional structures (such as for example the wells) can be formed, into which liquids with molecules for binding (molecules for coupling) can be introduced, so that the binding that actually takes place is concentrated in the area of the indentation and does not affect (for example undesired) areas of the molding.

As already indicated, on the basis of the inventive molding it is possible to use a large number of materials as the matrix material, which because of their chemistry have previously not been suitable for binding applications for biomolecules, at least not for binding applications without an additional coating.

A further advantage of the invention can be seen as being that, through the filler loading and the particle size, specific coupling densities of biomolecules on the inventive moldings can be set. On top of this it is possible, through the use of different coupling particles, thus coupling particles which have differing coupling chemistries, to specifically bind more than one species (or type of species) of biomolecules. Since the concentration of the various coupling particles can also be set separately in each case, it is thus also possible also to control the concentration of the loading of the surface of the inventive molding with specific biomolecules independently of one another, where the biomolecules have a different binding chemistry.

Furthermore, it is in particular possible for matrix materials in polymer synthetic materials, to considerably improve the ratio of specific to non-specific bindings: even if for the matrix material a synthetic material is selected which is not completely inert in relation to the biomolecules to be bound (or other biomolecules, to which the molding is exposed), through the specifically selected binding chemistry of the coupling particles a particularly high affinity to the desired biomolecules can be set, so that in areas in which the coupling particles are exposed through the mechanical treatment, a substantial enrichment of the biomolecules takes place.

For the manufacture of the molding a person skilled in the art will match the desired manufacturing method, the matrix material and the coupling particles, here in particular their size and/or form, to one another. There is extensive experience on this, e.g. in the area of injection molding.

An advantage of the preferred inventive molding is that through the use of synthetic materials which are often economically favorable, more expensive materials can be replaced: thus for example instead of solid glass surfaces, synthetic materials can be used as matrix materials, and glass particles as coupling particles. In addition to this, with synthetic material molding forms can be created which with, for example, glass are only possible with great effort, if at all.

Due to the fact that in the preferred inventive molding the matrix material and the coupling particles are positioned next to each other, it is possible to exploit all the advantages of synthetic materials for various purposes. At the same time it is possible to use widely developed and very often highly economical synthetic material manufacturing methods or synthetic material molding methods, in order to create the inventive moldings. A comparable approach is not known from the state of the art. U.S. Pat. No. 5,993,935 A does indeed describe a porous matrix with particles for molecular binding. With these matrices, however, it is a case of microporous membrane systems, which are not moldings in the sense of this invention. The woven or unwoven textiles described in said document cannot be understood to be moldings in the sense of the present invention either. In addition in this document there is no indication of any mechanical treatment of the matrix material to expose coupling particles.

Preferred inventive moldings are impervious to water, which ensures that the desired binding of the biomolecules takes place exclusively to the surface (and here preferably in the area where the moldings have been mechanically treated).

Preferred inventive moldings have a minimum thickness of 5 μm, preferably ≧100 μm, more preferably ≧1 cm and particularly preferably ≧10 cm.

Preference is for an inventive molding on which at least 1%, preferably ≧5%, more preferably ≧10% and particularly preferably ≧25% of an area of the molding is mechanically treated. At the same time it may be preferred that a maximum of 80%, preferably a maximum of 70%, more preferably a maximum of 50% of the surface of the molding is treated. The more area that is mechanically treated, the more coupling particles are exposed on the surface of the inventive molding. Accordingly the loading of the molding with biomolecules that can be achieved can also be controlled by the area that has been mechanically treated.

In this connection it should further be mentioned that the coupling particles used are often more expensive than the matrix material. Accordingly it is often desired that the molding has a high surface to volume ratio, so that the greatest possible proportion of coupling particles can be made available for binding. On the other hand a distribution of the coupling particles over the entire molding basically allows considerable simplifications in the manufacturing process, so that higher costs of material use of coupling particles can be offset by the advantages in the manufacture of the moldings.

Furthermore, a homogenous distribution of the coupling particles has the advantage that the mechanical treatment can also be introduced three-dimensionally into the molding (thus also have depth), so that for example indentations such as wells (see above) can be fashioned. This ensures that despite the three dimensional treatment an even concentration of binding points per unit of surface area is always available. With complicated three dimensional surface designs, for example, this cannot be ensured, or only with difficulty, if the binding points for the binding molecules are first applied to an additional coating on a body: it is not always easy to ensure complete coverage in three dimensional structures with a new layer.

Preference is for an inventive molding, that is mechanically treated in such a way that a proportion of the particles protrudes from the surface level of the molding. This is possible in that with the mechanical treatment the matrix material is removed in such a way that the coupling particles are not affected or only to a very minor extent. This leads to a higher surface area of the coupling particle being available for binding to the biomolecules. Of course it has to be ensured here that sufficient embedding of the coupling particles in the matrix material still exists.

In preferred inventive moldings the synthetic material matrix comprises material selected from synthetic materials consisting of thermoplastics, duroplastics and elastomers. For some applications it is preferred that the synthetic material matrix consists of this material.

The matrix material is preferably selected from the group consisting of polypropylene (PP), polystyrene (PS), polyurethane (PU), polycarbonate (PC), polymethyl methacrylate (PMMA), polyoxymethylene (POM), polyvinylchloride (PVC), polyethylene (PE), thermoplastic polyurethane (TPU), polyetheretherketone (PEEK), polytetrafluorethylene (PTFE) and biopolymers, in particular thermoplastic starch and PLA (polylactic acid).

Preference according to the invention is for a molding, wherein the coupling particles comprise a material or consist of a material, selected from the group consisting of metal, metal oxide, ceramic, glass and synthetic materials with a higher melting temperature than the matrix material.

Preferred materials for metals (as coupling particles) are in this connection gold (Au), cobalt (Co), zinc (Cn), tungsten, iron (Fe), copper (Cu), nickel (Ni), nickel, magnesium (Mg), aluminum (Al), titanium (Ti) or alloys of these such as for example steel or stainless steel. Said metals can also be considered as matrix metals.

Preferred materials for ceramics (as coupling particles) are ceramics based on calcium carbonate, aluminum oxide, hydroxylapatite, zirconium dioxide, titanium di-oxide, indium tin oxide (ITO), barium oxide or calcium phosphate.

Preferred glasses (as coupling particles) are silicon dioxide-based glass and silicate glass, which in the following are also referred to as glass balls or glass particles.

Preferred polymers (as coupling particles) are PC, PEEK, PMMA or may be selected from the list of matrix materials, where the combination takes into account a higher melting point of the coupling particles than the matrix. A large number of methods for surface modification are known which bring about the improvement of the coupling properties in respect of (certain) biomolecules. This applies in particular also for the above-mentioned preferred materials, which may be contained in coupling particles. Accordingly a preferred inventive molding comprises coupling particles which at least in the area where they are not covered by the matrix, are at least in part surface-modified in order to improve the coupling properties with respect to biomolecules.

Coupling particles means not only particles with a spherical, platelet, cylindrical or tubular appearance or structure, but also those with a fibrous appearance or structure.

In connection with the surface modification of the coupling particles, functional groups are preferably introduced. Preferred functional groups are, for example, epoxy, hydrazine, isocyanate, catechol and azlactone groups, as well as hydroxyl, sulfhydryl, amino or carboxyl groups on the coupling particle surface.

Here the surface modification can take place prior to manufacture of the molding. This means that in the manufacturing method for the inventive molding, surface modified coupling particles have already been introduced. It may also be preferable, however, to carry out a corresponding surface modification only after manufacture of the molding, e.g. if the chemicals to be used for modification are expensive. Then these need only be used in lower concentration, since they only have to be bound to the exposed areas of the coupling particles following mechanical treatment.

Preferred methods for surface modification of the coupling particles are silanization, in particular application of specific layers, with amino, epoxy or carboxyl silanes (including modified silanes), e.g. dihydroxyphenylalanine (DOPA), allowing binding of the biomolecules via carboxylic acids, amino, epoxy, hydroxyl, isocyanate, sugar, photoreactive or sulfhydryl groups. Here a person skilled in the art will of course select the suitable point in time for application of the binding chemistry (surface modification): where sensitive groups are required for the specific binding of the desired biomolecules, it would seem that the surface modification should be carried out only after manufacture of the inventive molding.

According to the invention it is preferred that the surface modification of the coupling particles (for the introduction of functional groups) especially after making the molding is made so that the modification is made on the coupling particles compared to the matrix material in a clearly higher amount, preferred exclusively. In a “clearly higher amount” means in this context the following: For introducing the functional groups/linkers during the surface modification, the coupling particles, the matrix material, the functional groups to be coupled to the coupling particles and/or the reaction conditions are chosen so that there are bound ≧50% preferred ≧75%, more preferred ≧99% of the functional groups per unit area to the coupling particles.

Preference according to the invention is for an inventive molding that is or can be manufactured using one or more methods such as injection molding methods or injection compression molding, by extrusion, hot stamping, stamping or molding, with subsequent mechanical treatment.

Particular preference in this connection is for the use of an injection molding method. The fact that the inventive molding can be manufactured using these common and economical methods is, particularly from the cost point of view, a considerable advantage of the invention. In particular, however, through these typical methods particularly simple three dimensional designs are possible and the methods are also tried and tested in mass production, so that large quantities of the inventive moldings can be created according to the invention.

Particularly preferred methods for the manufacture of inventive moldings are methods in which more than one component can be used. Here preferential mention is made of two- or multi-component injection molding. Here the components used in the multi-component method for manufacture of the inventive molding can be
a) one component, comprising coupling particles and another component comprising no coupling particles,
b) two components, each comprising a different coupling particle,
c) components, which comprise synthetic material matrix materials that are different from one another, and/or
d) components, which differ regarding their matrix material composition and regarding the coupling particles contained therein.

The advantage of this multi-component method can be considered to be that on the one hand the use of expensive materials (usually these will be the coupling particles) can be reduced, and on the other as required various zones within the inventive molding can be created which have different properties and different functionalizations. Here the combinations of the coupling particles and the matrix material within a molding can be matched to various functions of the sub-areas of a molding. So for example it is frequently unnecessary for coupling particles to be present inside the molding, since they perform their function on the surface. Thus with a corresponding design of the forming in the manufacturing method for the inventive molding, the material use for generally more expensive coupling particles can be reduced, in that for example a “core” of the molding is created in a component which is free from coupling particles. In addition various zones can be created on the inventive molding which according to the material combination of coupling particle and matrix material perform different functions, such as for example the binding of particular molecules.

Part of the invention is an inventive molding, comprising biomolecules coupled to its surface. These biomolecules are preferably selected from the group consisting of proteins, peptides, carbohydrates, lipids, nucleic acids, hormones, amino acids and nucleotides.

Biomolecules in the sense of the present invention are molecules which can be manufactured in organisms.

A corresponding preferred inventive molding, to which biomolecules are coupled, can fulfill a large number of functions. It can be used as a biosensor, for chemical, biochemical or immunological detection reactions, and in the area of qualitative and quantitative analysis, it can serve as sensor, as adhesion medium for cell cultures or as part of an implant. Basically such a preferred inventive molding allows a large number of applications which call for biomolecules on the surface.

Preference is for an inventive molding, which comprises biomolecules coupled to its surface, wherein ≧70% preferably ≧85%, more preferably ≧95% with particular preference for ≧99% of the biomolecules are coupled to the coupling particles, in relation to the quantity of all biomolecules coupled to the molding.

Such a preferred inventive molding is designed in such a way that a majority of the biomolecules to which it is exposed attach to the coupling particles. As already indicated, this can be ensured through a corresponding selection of the matrix material. In this way non-specific and/or undesired bindings to the molding can be avoided.

As already mentioned above, the molding can be designed in such a way that a large number of defined binding zones are created. Thus the mechanical treatment of the surfaces can take place in such a way that the binding zone takes the form of wells, which are not in contact with each other. In this way it is possible to create separate reaction spaces, each of which can be loaded with the same or different biomolecules. In turn each of these separately can be exposed to a specimen, which for each reaction space has an identical composition or which can differ from reaction space to reaction space. Thus mass testing—which may also be automated—is eminently possible. This system can be made more flexible by applying to the coupling particles in the individual reaction spaces binding groups which differ from those on the particles in other reaction spaces.

Preference is for an inventive molding, to which the biomolecules are coupled via complexing or covalently. In this connection it is obviously preferable for a majority of the biomolecules to be coupled to the coupling particles. This preferred form of coupling leads to a durable binding, which is necessary for many applications. Furthermore, in principle it is possible to control the number of binding points through suitable methods, such as by creating a particular loading density with reactive groups on the coupling particles, as well as allowing targeted coupling.

Part of the invention is also a method for manufacturing an inventive molding, comprising the following steps:

a) provision of a synthetic material and/or a synthetic precursor material and/or a metal material and/or a ceramic precursor material,
b) provision of coupling particles,
c) mixing of the coupling particles with the synthetic material and/or with the synthetic precursor material and/or with the metal material and/or with the ceramic precursor material,
d) forming of a molding from the mixture,
e) if necessary in the case of metals and ceramics, burning out of any polymers or waxy binding materials (debinders),
f) if necessary in the case of metals and some ceramics, sintering for hardening and compressing the matrix material at high temperatures,
g) and mechanical treatment of the molding so that a proportion of the surface of the molding in a geometrical form or in a regular pattern and/or an area of the molding is completely or the entire surface of the molding is mechanically treated.

A preferred inventive method for manufacturing an inventive polymer molding comprises here the following steps:

a) provision of a synthetic material and/or a synthetic precursor material (e.g. monomers or oligomers) for a synthetic material matrix,
b) provision of coupling particles,
c) mixing of the coupling particles with the synthetic material and/or with the synthetic precursor material (e.g. monomers or oligomers),
d) forming of a molding from the mixture, and
e) mechanical treatment of the molding so that a proportion of the particle is at least in part not covered by the synthetic material matrix, and a proportion of the surface of the molding in a geometrical form or in a regular pattern and/or an area of the molding is completely or the entire surface of the molding is mechanically treated.

An advantage of the inventive method is that through a corresponding material composition of the matrix material, in particular synthetic materials and coupling particles, inventive moldings can be manufactured which are suitable for a large number of bindings of different kinds. Here preferably synthetic material or the synthetic precursor material is used as matrix material.

Also preferred is an inventive method, wherein after the mechanical treatment the coupling of biomolecules to coupling particles which are at least in part not covered by the synthetic matrix takes place. Here the coupling can preferably take place in different regions of the inventive molding through biomolecules that differ from one another.

Part of the invention is also the use of an inventive molding as sensor, biochip, for diagnostics purposes, for immunological detection methods, for as bioreactor or component of a bioreactor, for lab-on-a-chip applications, for cleaning of mixtures containing biomolecules, for testing substances for impurities, for the generation of or as a biocatalytic and/or bioactive surface, for cell structure purposes, for manufacturing an implant. The inventive moldings can be used for manufacture in a number of applications thanks to their adaptability. These include detection methods of all kinds, wherein the coupling particles can be matched with the molecules to be detected or loaded with molecules such as antibodies, in order to be able to perform certain detection reactions. However, they can also be used for cleaning mixtures in which the coupling particles are designed in such a way that they bind certain impurities from the mixtures. Finally a large number of further usage possibilities for the inventive moldings exist for a person skilled in the art.

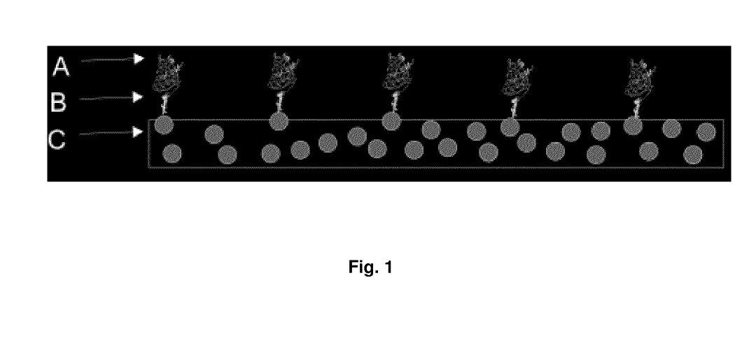

FIG. 1 shows a schematic representation of an inventive molding, in which A designates a biomolecule, which is bound via a specific group B to a coupling particle C. Examples of suitable combinations of biomolecule, specific group and coupling particle are shown in Table 1 below.

TABLE 1

| A - Biomolecule | B - Specific group | C - Coupling particle |
|---|---|---|
| Proteins (e.g. | Complex chemistry over, for | Nickel (Ni), cobalt |
| antibodies) | example histidine tag (his | (Co), copper (Cu) |
| | tag) but also over cysteine, | or zinc (Zn) |
| | tryptophan or phosphate | |
| | groups | |
| Peptides | Aminosilanes, epoxysilanes, | Oxides, e.g. glass |
| | carboxysilanes, with and | (SiO₂), Al₂O₃, ZrO₂, |
| | without polyethylene glycol | CaCO₃, ITO |
| | (PEG) or jeffamine-based | |
| | spacers | |
| Nucleic acids | Cysteines or over | Gold |
| | dithiodipropionic acid | |
| | (DTPA) | |
| Lipids | Dihydroxyphenylalanine | Iron particles |
| | (DOPA) | |
| Carbohydrate | Specific protein interaction | Magnesium, calcium |
| | (with for example integrins) | |
| Medicines | Carboxylic acids, e.g. in the | Acrylic particles, |
| | acrylic acid (in the acrylic) | e.g. PMMA |
| Proteins or | Avidin or streptavidin | Biotinylated polymer |
| peptides | | particles, e.g. |
| | | from polylactic acid |
| | | and derivatives or |
| | | co-polymerizates |
| | | thereof. |

Further examples for coupling of biomolecules to surfaces via covalent chemical bindings are listed in Willner and Katz (Angew. Chem. 2000, 112, 1230-1269).

Here it is clear to a person skilled in the art that the specific Group B can also be a component of the biomolecule, as for example may be the case with the histidine tag of a protein.

A person skilled in the art will adapt the particle form and the particle size to the respective purpose. The preferred maximum diameters for the coupling particles are in the range from 10 nm to 40 μm, more preferably from 50 nm to 10 μm, and particularly preferably from 100 nm to 5 μm.

### Example 1

### Manufacture of Moldings

For the coupling of biomolecules to solid centers (coupling particles) in the first step a composite is manufactured from the respective solid in powder form and a thermoplastic polymer. Manufacture takes place in a temperature-controlled mixer, which heats the thermoplastics to above the heat distortion temperature and plasticizes them through mechanical working. For the manufacture of the composites used a Brabender Plastograph was employed.

For the dispersion the thermoplastic was placed in the pre-heated mixer and plasticized through mechanical shearing forces. Then the coupling particles were added dry as an ultrafine powder. After a mixing time of approximately 60 minutes the material was cooled slowly. The result was a granulate.

For the manufacture of the material composite, various material combinations were considered. For the base synthetic material, polypropylene (PP), polystyrene (PS), polyethylene (PE) and polyurethane (PUR) were available. Similarly, the materials polycarbonate (PC) and polymethyl methacrylate (PMMA) were used. As filler particles (coupling particles) metallic particles (nickel), ceramic particles (SiO2) and glass balls were introduced into the synthetic materials listed.

Table 2 shows an overview of the trials performed:

| | | | | Quantity of | |
|---|---|---|---|---|---|
| | | | | coupling | |
| | | Particle | | particles (as a | |
| | | size | | proportion of | |
| | Filler | (average | | total mass/total | Unit |
| Matrix material/ | (coupling | diameter) | | volume of | Total |
| solvent | particles) | [μm] | Name | granulate) | mass |
| Lupolen (PE) | Ni | 6.8 | Novamet | 1% | % by |
| | | | | | weight |
| Lupolen (PE) | Ni | 2.3 | Fritsch | 1% | % by |
| | | | | | weight |
| Lupolen (PE) | Ni | 2.3 | Fritsch | 5% | % by |
| | | | | | weight |
| Lupolen (PE) | Ni | 2.3 | Fritsch | 10% | % by |
| | | | | | weight |
| PP | Ni | 2.3 | Fritsch | 5 | % by |
| | | | | | weight |
| PP | Ni | 2.3 | Fritsch | 10% | % by |
| | | | | | weight |
| PS | Ni | 2.3 | Fritsch | 5% | % by |
| | | | | | weight |
| PS | Ni | 2.3 | Fritsch | 10% | % by |
| | | | | | weight |
| Lupolen (PE) | Glass (S38) | 30 μm | 3M | 5% | % by |
| | | | | | volume |
| Lupolen (PE) | Glass (S38) | 30 μm | 3M | 10% | % by |
| | | | | | volume |
| PP | Glass (S38) | 30 μm | 3M | 5% | % by |
| | | | | | volume |
| PP | Glass (S38) | 30 μm | 3M | 10% | % by |
| | | | | | volume |
| PS | Glass (S38) | 30 μm | 3M | 10% | % by |
| | | | | | volume |
| PP | None | | Reference | 0% | |
| | | | sample | | |
| PUR1180A | Glass (S38) | 30 μm | | 10% | % by |
| | | | | | volume |
| PUR1180A | Ni | 2.3 | Fritsch | 10% | % by |
| | | | | | weight |
| PUR685A | Glass (S38) | 30 μm | | 10% | % by |
| | | | | | volume |
| PUR685A | Ni | 2.3 | Fritsch | 10% | % by |
| | | | | | weight |
| Lupolen (PE) | Ni | 2.3 | Novamet | 10% | % by |
| | | | | | weight |
| Lupolen (PE) | Aerosil | 14 nm | Plasma- | 1% | % by |
| | | | Chem | | volume |
| Lupolen (PE) | Aerosil | 14 nm | Plasma- | 5% | % by |
| | | | Chem | | volume |
| Adisil Rapid | SiO₂ | | Aerosil 200 | 3% | % by |
| | | | | | volume |
| Adisil Rapid | SiO₂ | | Aerosil 200 | 2.5% | % by |
| | | | | | volume |

Explanations: Glass (S38) means glass balls from 3M, Fritsch stands for Dr. Fritsch GmbH & Co KG.

With the granulate obtained, conventional injection molding machines can now be used to produce moldings. The test specimens were manufactured using a laboratory injection molding machine from MCP HEK Tooling GmbH.

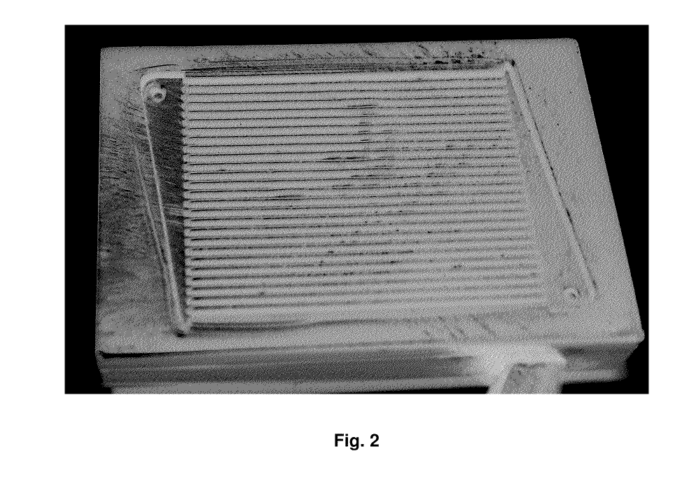

Various geometries were used for the tool shape. Trials on the attachment to nickel were generally carried out with the “photoreactor” mold part which is shown in FIG. 2.

For the “photoreactor” geometry the following material combinations were created:

TABLE 3
Photoreactor material combinations

| | | Gehalt des | | | |
|---|---|---|---|---|---|
| | | Füllmaterials | Temperatur | Zeit | |
| | | Ni [Gew %] | Mischen | Mischen | Temperatur |
| Kunststoff | Füllmaterial | Glas [Vol %] | [° C.] | [min] | Spritzgu β [° C.] |
| PS | X | X | 175.00 | 60 | 175.00 |
| PS | Glas | 5 | 175.00 | 60 | 175.00 |
| PS | Glas | 10 | 175.00 | 60 | 175.00 |
| PS | Nickel | 5 | 175.00 | 60 | 175.00 |
| PS | Nickel | 10 | 175.00 | 60 | 175.00 |
| PP | X | X | 170.00 | 60 | ca. 180° C. |
| PP | Glas | 5 | 170.00 | 60 | ca. 180° C. |
| PP | Glas | 10 | 170.00 | 60 | ca. 180° C. |
| PP | Nickel | 5 | 170.00 | 60 | ca. 180° C. |
| PP | Nickel | 10 | 170.00 | 60 | ca. 180° C. |
| PE | X | X | 120 | 60 | 135.00 |
| PE | Glas | 5 | 120 | 60 | 135.00 |
| PE | Glas | 10 | 120 | 60 | 135.00 |
| PE | Nickel | 5 | 120.00 | 60 | 135.00 |
| PE | Nickel | 10 | 120.00 | 60 | 135.00 |
| PE Arburg | X | X | X | X | 120.00 |
| PC | X | X | X | X | 230.00 |
| PMMA | X | X | 220.00 | 60 | 220.00 |
| PUR 1180 | X | X | 210.00 | 60 | 185.00 |
| PUR 1180 Arburg | X | X | X | X | 210.00 |
| PUR 685 | X | X | X | X | 185.00 |
| PUR685 | X | X | X | X | 185.00 |
| PUR685Arburg | X | X | X | X | 205.00 |

### [Column Headings, Left to Right:]

Synthetic material
Filler material
Content of filler material Ni [% by weight] Glass % by volume
Mixing temperature
Mixer time [minutes]
Injection molding temperature [° C.]

### Glas=Glass

### Example 2

### Moldings

As a further geometry, in the injection molding method, spherical-cylindrical moldings were manufactured. Table 4 gives an overview of the material combinations produced:

TABLE 4
Material combination for spherical-cylindrical moldings

| | | Content of | | | | | Dwell |
|---|---|---|---|---|---|---|---|
| | | coupling | | | | | pressure |
| | | particles | Mixing | Mixer | Injection | Air | for 30 |
| Synthetic | Coupling | [% by | temperature | time | molding | pressure | second |
| material | particle | volume] | [° C.] | [min] | temperature | [bar] | setting |
| PE | X | X | 125 | 15 | 125 | 4 | 0.5 |
| PE | X | X | X | X | 125 | 4 | 0.5 |
| PE | Glass | 10 | 125 | 15 | 128-130 | 4 | 0.5 |
| | (S38) | | | | | | |
| PE | Nickel | 10 | 125 | 15 | 128-130 | 4 | 0.5 |
| | (Novamet) | | | | | | |

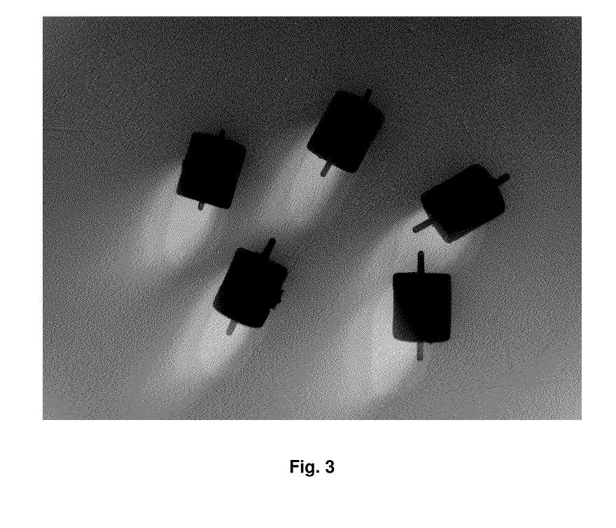

FIG. 3 shows injection molded parts (moldings) with 10% by volume of glass balls (average particle diameter of 20 μm) in a polyethylene matrix.

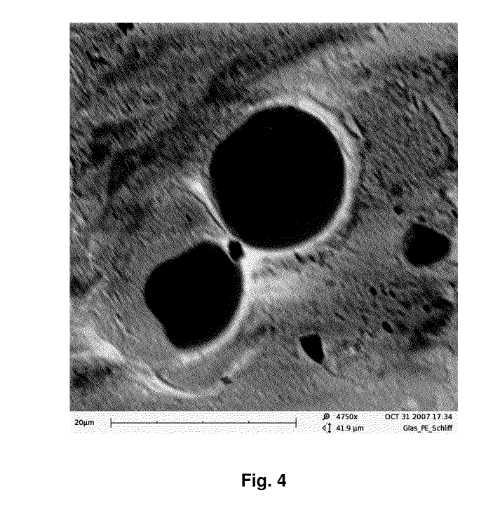

For the biochemical reaction (coupling) the active centers (glass balls) must now be exposed. This can take place by means of various methods, such as for example chemical or plasma-chemical etching. With these methods, however, often new unselective active centers in the form of carboxyl or similar groups are activated on the surface of the polymers, which promote the coupling of biomolecules to the entire polymer surface. In order to avoid this surface activation a mechanical removal method was selected for exposing the particles on the composition surface. The composite specimens were in each case ground for approximately 5 minutes on a grinding and polishing machine of the Stuer LaboPol-21 type with a grain size of between P500 and P1200. Following the treatment the filler particles were exposed on the surface as can be seen from FIG. 4.

FIG. 4 shows the surface of a molding of ten percent by volume of glass balls, which have been introduced into a polyethylene matrix. Two exposed glass balls with a diameter of less than 15 μm can clearly be noticed.

### Application Example 1

### Polyethylene Bars with Silanized ITO Particles

The covalent coupling of biomolecules to fixed particles in polymers offers the possibility of creating a biochemical sensor. For this, however, another evaluation or recognition of the coupling process is necessary. A very simple and at the same time effective possibility is offered by electrical evaluation of surface changes or surface potentials. A necessary basic condition for this is a sufficiently high electrical conductivity of the specimen. Since the coupling of the biomolecules is to take place with the coupling particles introduced, these particles should demonstrate sufficient electrical conductivity. As a coupling mechanism the binding of a silane to an oxide layer was selected. The necessary properties set out here bind semi-conductive oxides, such as for example indium tin oxide ITO. Through the combination of such oxides in an electrical non-conducting polymer such as for example polyethylene, the possibility arises of measuring a covalent coupling of biomolecules to the active oxide particles through the variation in electrical parameters. For the transmission of the electrical signals from the surface of the composite as far as the evaluation electronics an electrical conductivity of the composite is necessary. This can be achieved by varying the filler content. For concentrations in the range of approximately 20 to 30% by volume percolation results in such composites. Above this concentration through the statistical distribution of the particles in the matrix coherent areas (clusters) of particles result, which are connected together through points of contact. Via these clusters (of electrically semi-conducting particles) the electrical information can be transmitted through the entire composite. The necessary concentration for percolation is dependent upon the particle geometry, the material and other factors, so that the necessary concentration must be determined anew for each system. With this electrically conductive composite the reaction mechanisms to be investigated can now be measured electrically. This can take place, for example, by means of impedance measurement or cyclic voltammetry. At the same time the only extremely low conductivity of the pure polymer prevents the recording of undesired couplings between biomolecules and the polymer surface.

In order to test this application example, in a PE with 30% by volume ITO powder was mixed. The resultant composite material was processed via a mini-extruder to form flat bars. The coupling trials showed (see application example 2), that with the moldings treated with aminosilane and the biomolecule CF-Ala (carboxidifluoresceine-alanine) a signal could be seen in the fluorescence microscope. The controls without aminosilane, but with biomolecule CF-Ala showed no signal, since the coupling particles were not provided with a reactive function.

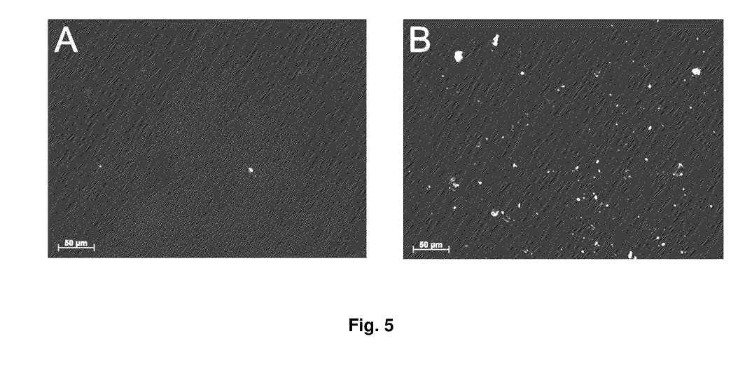

FIG. 5 shows fluorescent microscope images of polyethylene moldings, manufactured according to application example 1. Here for the molding shown in FIG. 5 A, a silane functionalization was dispensed with. The moldings shown in FIG. 5 B on the other hand comprise aminosilane-functionalized particles. It can be clearly seen that on the molding in Image A no coupling of the biomolecule CF-Ala took place, while in Image B clear signals of the coupled biomolecules can be detected. Images were taken with a fluorescence microscope (Axio Imager.M1 from Zeiss) and then converted to SW.

### Application Example 2

### Coupling of Carboxyfluorescein-Alanine to Moldings

Using the solid phase peptide synthesizer a carboxidifluorescein molecule was coupled with four alanine molecules. This coupled molecule (CF-Ala) was to be coupled as a biomolecule specimen to inventive moldings. For this purpose the spherical-cylindrical moldings from Example 2 were used as moldings, in the material combination described in Table 4 of polyethylene with 10% by volume glass (S 38) as coupling particles. As a control, spherical-cylindrical moldings in PE containing no coupling particles were used.

As the next step all spherical-cylindrical moldings underwent silanization with aminosilane. For this purpose spherical-cylindrical moldings were treated with a 2% (v/v) solution of 3-aminopropyltrimethoxysilane (APTMS) in acetone. The silanization took place for 30 seconds. Following incubation the silanization solution was sucked out and the spherical-cylindrical moldings washed with 100% acetone and then dried with nitrogen. The coupling of the CF-Ala to the spherical-cylindrical moldings was carried out with 1-ethyl-3-(3-dimethylaminopropyl)-carbodiimide (EDC, 10 mg/ml in 2-(N-morpholino)ethanesulfonic acid (MES) buffer) as the crosslinker and N-hydroxysuccinimide (NHS, 0.6 mg/ml in MES buffer), as the stabilizer of the intermediate product of EDC-activated CF-Ala (1 mg/ml in MES Puffer). The incubation took place for 2 hours at room temperature on an orbital shaker. Then the spherical-cylindrical moldings were washed for one hour with phosphate-buffered saline solution with Tween 100 (PBS-T), dried and observed under a fluorescence microscope (Carl Zeiss Axio Imager M1, filter set F9, illumination time 15 milliseconds).

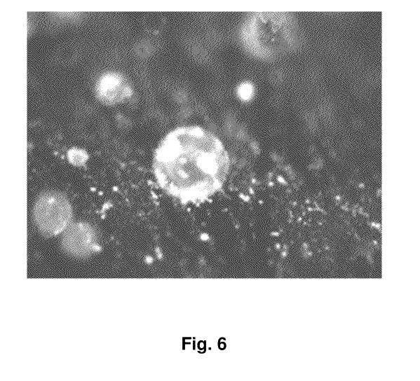

FIG. 6 shows a fluorescence microscopy image of the molding according to application example 2. The image was taken following coupling of CF-alanine to the coupling particles. Here the embedded particles can be clearly seen as a result of the biofunctionalization (coupling of CF-alanine) that has taken place in the fluorescence mode of the microscope.

Here the surfaces of the spherical-cylindrical moldings (which had been ground prior to silanization) were exposed to a solution of CF-Ala in ethanol for 15 minutes and 30 minutes. As a result, a distinctly raised coupling concentration of CF-Ala on the spherical-cylindrical moldings provided with coupling particles could be detected. The increase in concentration of the coupled biomolecules here was based on a significantly increased molecule density in the region of the coupling particles.

### Application Example 3

### Coupling to Nickel Coupling Particles

Moldings manufactured according to Example 1 of the photoreactor type in polyethylene (Lupolen) as the matrix material and 5% by weight nickel particles with an average particle diameter of 2.3 μm (Fritsch) as coupling particles were exposed to a solution of the protein TNFalpha, provided with a histidine tag. The matrix material used without nickel particles served as a control. Mechanical removal by grinding was applied in each case to the moldings. The moldings with and without nickel particles were incubated with a TNFalpha solution of 10 mg/ml in phosphate buffered saline solution (PBS) for 3 hours at ambient temperature on a tilting shaker. Then excess or unbound protein was treated with a washing solution (PBS with 0.1% [v/v] of the detergent Tween 100). The bound protein TNFalpha was then detected by an antibody-mediated enzyme test, as this is carried out in the Enzyme-Linked Immunoabsorbent Sandwich Essay (ELISA) known from the state of the art. Here the TNFalpha was detected with an anti-TNF antibody, which was coupled with the enzyme peroxidase. The detection took place through the addition of the substance tetramethylbenzidine (TMB). The TMB is converted from a colorless substance by the peroxidase into a blue dye. In the process, by means of the enzyme-coupled antibody detection, it was possible to demonstrate that addition of the his-tag-bound proteins took place almost exclusively on the nickel particles of the molding filled with nickel. On the control molding only a very weak non-specific binding was observed.

1. A molding, comprising a matrix in a material, selected from the group consisting of metal, ceramic and polymer synthetic material, and coupling particles embedded in the matrix, wherein a proportion of the surface of the molding in a geometrical form or in a regular pattern and/or an area of the molding is completely or the entire surface of the molding is mechanically treated.

2. The molding, as claimed in claim 1, wherein the matrix is a polymer synthetic material matrix and the molding is mechanically treated, so a proportion of the particle is at least in part not covered by the synthetic material matrix.

3. The molding as claimed in claim 1, wherein the mechanical treatment is performed by grinding, milling and/or sand blasting.

4. The molding as claimed in claim 1, wherein the molding is impervious to water.

5. The molding as claimed in claim 1, wherein at least 1% of the area of the molding is mechanically treated.

6. The molding as claimed in claim 1, wherein the molding is mechanically treated in such a way that a proportion of the particle protrudes from the surface level of the molding and is partially exposed.

7. The molding as claimed in claim 1, wherein the synthetic material matrix comprises material selected from the group consisting of thermoplastics, duroplastics and elastomers.

8. The molding as claimed in claim 1, wherein the matrix material is selected from the group consisting of polypropylene PP, polystyrene PS, polyurethane PU, polycarbonate PC, polymethylmethacrylate PMMA, polyoxymethylene POM, polyvinylchloride PVC, polyethylene PE, thermoplastic polyurethane TPU, polyetheretherketone PEEK, polytetrafluorethylene PTFE and biopolymers, in particular thermoplastic starch and (polylactic acid).

9. The molding as claimed in claim 1, wherein the coupling particles comprise a material or consist of a material selected from the group consisting of metal, metal oxide, ceramic, glass and synthetic materials with a higher melting temperature than the matrix material.

10. The molding as claimed in claim 1, wherein the coupling particles at least in the area where they are not covered by the matrix, at least in part to improve the biomolecule coupling properties are surface-modified.

11. The molding as claimed in claim 10, wherein the surface modification comprises or consists of the introduction of functional groups.

12. The molding as claimed in claim 1, that is or can be manufactured using or by means of an injection compression molding method, by extrusion, hot stamping, stamping or molding with subsequent mechanical treatment.

13. The molding as claimed in claim 1, comprising biomolecules coupled to its surface.

14. The molding as claimed in claim 1, wherein the biomolecules are selected from the group consisting of proteins, peptides, carbohydrates, lipids, carbohydrates, nucleic acids, hormones, amino acids and nucleotides.

15. The molding as claimed in claim 13, wherein ≧1%, preferably ≧85%, more preferably ≧95% with particular preference for ≧99% of the biomolecules are coupled to the coupling particles.

16. The molding as claimed in claim 13, wherein the biomolecules are coupled via complexing or covalently.

17. A method for manufacturing a molding as claimed in claim 1, comprising the following steps:
a) provision of a synthetic material and/or a synthetic precursor material and/or a metal material and/or a ceramic precursor material,
b) provision of coupling particles,
c) mixing of the coupling particles with the synthetic material and/or with the synthetic precursor material and/or with the metal material and/or with the ceramic precursor material,
d) forming of a molding from the mixture
e) mechanical treatment of the molding so that a proportion of the surface of the molding in a geometrical form or in a regular pattern and/or an area of the molding is completely or the entire surface of the molding, is mechanically treated.

18. The method as claimed in claim 17, wherein in step a) a synthetic material and/or a synthetic precursor material is provided and step d) takes place in such a way that a proportion of the particles is at least in part not covered by the synthetic material matrix.

19. The method as claimed in claim 17, wherein after mechanical treatment, coupling of biomolecules to coupling particles which are at least in part not covered by the matrix takes place.

20. A use of a molding as claimed in claim 1 as sensor, biochip, for diagnostic purposes for immunological detection methods, as bioreactor or component of a bioreactor, for lab-on-a-chip applications, for cleaning mixtures containing biomolecules, for testing substances for impurities, for generation of or as a biocatalytic and/or bioactive surface, for cell culture purposes, for cell biology and immunological investigations on biofunctionalized moldings, for manufacturing an implant or a biofunctionalized medical device.

21. The molding as claimed in claim 2, wherein:
the mechanical treatment is performed by grinding, milling and/or sand blasting;
the molding is impervious to water;
at least 1% of the area of the molding is mechanically treated;
the molding is mechanically treated in such a way that a proportion of the particle protrudes from the surface level of the molding and is partially exposed;
the synthetic material matrix comprises material selected from the group consisting of thermoplastics, duroplastics and elastomers;
the matrix material is selected from the group consisting of polypropylene PP, polystyrene PS, polyurethane PU, polycarbonate PC, polymethylmethacrylate PMMA, polyoxymethylene POM, polyvinylchloride PVC, polyethylene PE, thermoplastic polyurethane TPU, polyetheretherketone PEEK, polytetrafluorethylene PTFE and biopolymers, in particular thermoplastic starch and (polylactic acid);
the coupling particles comprise a material or consist of a material selected from the group consisting of metal, metal oxide, ceramic, glass and synthetic materials with a higher melting temperature than the matrix material;
the coupling particles at least in the area where they are not covered by the matrix, at least in part to improve the biomolecule coupling properties are surface-modified;
the surface modification comprises or consists of the introduction of functional groups;
the molding is or can be manufactured using or by means of an injection compression molding method, by extrusion, hot stamping, stamping or molding with subsequent mechanical treatment;
biomolecules are coupled to its surface;
the biomolecules are selected from the group consisting of proteins, peptides, carbohydrates, lipids, carbohydrates, nucleic acids, hormones, amino acids and nucleotides;
≧1%, preferably ≧85%, more preferably ≧95% with particular preference for ≧99% of the biomolecules are coupled to the coupling particles; and
the biomolecules are coupled via complexing or covalently.

22. A method for manufacturing a molding as claimed in claim 19, comprising the following steps:
a) provision of a synthetic material and/or a synthetic precursor material and/or a metal material and/or a ceramic precursor material,
b) provision of coupling particles,
c) mixing of the coupling particles with the synthetic material and/or with the synthetic precursor material and/or with the metal material and/or with the ceramic precursor material,
d) forming of a molding from the mixture,
e) mechanical treatment of the molding so that a proportion of the surface of the molding in a geometrical form or in a regular pattern and/or an area of the molding is completely or the entire surface of the molding, is mechanically treated, wherein:
in step a) a synthetic material and/or a synthetic precursor material is provided and step d) takes place in such a way that a proportion of the particles is at least in part not covered by the synthetic material matrix; and
wherein after mechanical treatment, coupling of biomolecules to coupling particles which are at least in part not covered by the matrix takes place.
